(19) United States
(12) Patent Application Publication
(10) Pub. No.: US 2010/0114144 A1
(43) Pub. Date: May 6, 2010

(54) INTRA-GASTRIC SATIETY CREATION DEVICE WITH DATA HANDLING DEVICES AND METHODS

(76) Inventors: Thomas E. ALBRECHT, Cincinnati, OH (US); Jason L. Harris, Mason, OH (US); Mark S. ORTIZ, Milford, OH (US); Mark S. ZEINER, Mason, OH (US); Daniel F. Dlugos, JR., Middletown, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,093

(22) Filed: Oct. 30, 2008

(51) Int. Cl. A61M 29/02

(52) U.S. Cl. 606/191

(57) ABSTRACT

An implant for placement within a hollow body organ. The implant includes an implantable distension device having an undeployed shape for delivery within a hollow body and one or more deployed shapes for implantation therein. The implantable distension device has sufficient rigidity in its deployed shape to exert an outward force against an interior of the hollow body so as to bring together two substantially opposing surfaces of the hollow body. The implant includes a powered means for changing the deployed shape of the implantable distension device while implanted within the hollow body. The implant also includes an implantable sensing device in communication with the implantable distension device and configured to sense a parameter related to the implantable distension device and to communicate the parameter to a filter, the filter transmits a selected portion of the parameter to a data storage device.

This case is related to the following commonly assigned and concurrently filed U.S. Applications, all of which are hereby incorporated herein by reference:

U.S. Ser. No. ______ (Attorney Docket Number END6514USNP) titled DEVICES and METHODS FOR ADJUSTING A SATIATION AND SATIETY-INDUCING IMPLANTED DEVICE; U.S. Ser. No. ______ (Attorney Docket Number END6515USNP) titled Sensor Trigger; U.S. Ser. No. ______ (Attorney Docket Number END6516USNP) titled AUTOMATICALLY ADJUSTING INTRA-GASTRIC SATIATION AND SATIETY CREATION DEVICE; U.S. Ser. No. ______ (Attorney Docket Number END6517USNP) titled OPTIMIZING THE OPERATION OF AN INTRA-GASTRIC SATIETY CREATION DEVICE; U.S. Ser. No. ______ (Attorney Docket Number END6518USNP) titled POWERING IMPLANTABLE DISTENSION SYSTEMS USING INTERNAL ENERGY HARVESTING MEANS; U.S. Ser. No. ______ (Attorney Docket Number END6519USNP) titled WEARABLE ELEMENTS FOR INTRA-GASTRIC SATIETY CREATION SYSTEMS; U.S. Ser. No. ______ (Attorney Docket Number END6520USNP) titled INTRA-GASTRIC SATIETY CREATION DEVICE WITH DATA HANDLING DEVICES AND METHODS; U.S. Ser. No. ______ (Attorney Docket Number END6521USNP) titled GUI FOR AN IMPLANTABLE DISTENSION DEVICE AND A DATA LOGGER; U.S. Ser. No. ______ (Attorney Docket Number END6522USNP) titled METHODS AND DEVICES FOR FIXING ANTENNA ORIENTATION IN AN INTRA-GASTRIC SATIETY CREATION SYSTEM; U.S. Ser. No. ______ (Attorney Docket Number END6523USNP) titled METHODS AND DEVICES FOR PREDICTING INTRA-GASTRIC SATIETY CREATION DEVICE SYSTEM PERFORMANCE; U.S. Ser. No. ______ (Attorney Docket Number END6524USNP) titled CONSTANT FORCE MECHANISMS for Regulating Distension Devices; U.S. Ser. No. ______ (Attorney Docket Number END6525USNP) titled A METHOD OF REMOTELY ADJUSTING A SATIATION AND SATIETY-INDUCING IMPLANTED DEVICE.

### FIELD OF THE INVENTION

The present invention relates to devices and methods for handling data related to implantable distension devices.

### BACKGROUND OF THE INVENTION

Obesity is becoming a growing concern, particularly in the United States, as the number of obese people continues to increase and more is learned about the negative health effects of obesity. Morbid obesity, in which a person is 100 pounds or more over ideal body weight, in particular poses significant risks for severe health problems. Accordingly, a great deal of attention is being focused on treating obese patients. One proposed method of treating morbid obesity has been to place a distension device, such as a, spring loaded coil inside the stomach. Examples of satiation and satiety inducing gastric implants, optimal design features, as well as methods for installing and removing them are described in commonly owned and pending U.S. patent application Ser. No. 11/469,564, filed Sep. 1, 2006, and pending U.S. patent application Ser. No. 11/469,562, filed Sep. 1, 2006, which are hereby incorporated herein by reference in their entirety. One effect of the distension device is to more rapidly induce feelings of satiation defined herein as achieving a level of fullness during a meal that helps regulate the amount of food consumed. Another effect of the distension device is to prolong the effect of satiety which is defined herein as delaying the onset of hunger after a meal which in turn regulates the frequency of eating. By way of a non-limiting list of examples, positive impacts on satiation and satiety may be achieved by an intragastric distension device through one or more of the following mechanisms: reduction of stomach capacity, rapid engagement of stretch receptors, alterations in gastric motility, pressure induced alteration in gut hormone levels, and alterations to the flow of food either into or out of the stomach.

With each of the above-described stomach distension devices, safe, effective treatment requires that the device be regularly monitored and adjusted to vary the degree of distension applied to the stomach.

During these distension device adjustments, it may be difficult to determine how the adjustment is proceeding, and whether the adjustment will have the intended effect.

Additionally, it can be advantageous to acquire data indicating the pressure in a distension device before, during, and/or after pressure adjustment for adjustment, diagnostic, monitoring, or other purposes. It can be further advantageous to store such pressure data and/or communicate it to an external location. However, data storage space can be limited, and power to communicate data can be resource-intensive.

Accordingly, methods and devices are provided for use with a gastric distension device, and in particular for handling data gathered in relation to a gastric distension device.

### BRIEF DESCRIPTION OF THE DRAWINGS

The invention will be more fully understood from the following detailed description taken in conjunction with the accompanying drawings, in which:

FIG. 1A is a perspective view of an embodiment of a stomach distension system;

FIG. 1B is a side view of an embodiment of an implantable portion of the stomach distension system of FIG. 1A;

FIG. 2A is a cross sectional view of the stomach distension device of FIG. 1A;

FIG. 2B is a schematic diagram of the stomach distension device of FIG. 2A applied about within the stomach of a patient;

FIG. 3 is a perspective view of an embodiment of the injection port housing of FIG. 1A;

FIG. 4 is a side view of an embodiment of the sensor housing of FIG. 1A;

FIG. 5 illustrates an embodiment of the sensor housing of FIG. 1A;

FIG. 6 is a schematic of an embodiment of a variable resistance circuit for the pressure sensor of FIG. 5;

FIG. 7 is a block diagram showing an embodiment of internal and external components of the stomach distension device of FIG. 1A;

FIG. 8 is a flow diagram showing an embodiment of a data handling protocol for the stomach distension device of FIG. 1A;

FIG. 9 is a graphical representation of a pressure measurement from the pressure sensor of FIG. 5;

FIG. 10 is a graphical representation of another pressure measurement from the pressure sensor of FIG. 5;

FIG. 11 is a schematic diagram of an embodiment of a data logger for recording pressure measurements related to the stomach distension device of FIG. 1A;

FIG. 12 is a block diagram showing an embodiment of components of the data logger of FIG. 11;

FIG. 13 is a schematic diagram of an embodiment of a data logging system for recording pressure measurements related to the stomach distension device of FIG. 1A;

FIG. 14 is a is a block diagram showing an embodiment of components of the data logging system of FIG. 13;

FIG. 15 is a perspective view of an embodiment of a stomach distension system with a sensor positioned along a catheter;

FIG. 16 is a schematic view of an embodiment of a stomach distension system with a sensor positioned within a catheter;

FIG. 17 is a perspective view of another embodiment of a stomach distension system with a sensor positioned along a catheter; and

FIG. 18 is a schematic view of an embodiment of a gastric coil system with a “T”-shaped sensor and catheter configuration.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides devices and methods for handling data related to implantable distension devices. The distension device may also be adjustable. Exemplary non-limiting examples of adjustable implantable distension devices (e.g., satiation and satiety inducing gastric implants), optimal design features, as well as methods for installing and removing them are described in commonly owned and pending U.S. patent application Ser. No. ______, filed on even date herewith and entitled “Devices and Methods for Adjusting a Satiation and Satiety-Inducing Implanted Device” [Atty. Docket No. END6514USNP], which is hereby incorporated herein by reference in its entirety. In one embodiment, a distension system for forming a distension in a patient is provided that includes an implantable distension device that can cause a distension in a patient and an implantable sensing device in communication with the implantable distension device. The implantable sensing device can sense a clinically relevant parameter related to the implantable distension device and communicate a selected portion of data to an external device considering a variation of data from a nominal parameter value related to the implantable distension device. By way of a non-limiting list, the parameter can include at least one of, for example, stomach pH, pressure, pulse count, pulse width, and amplitude. In some embodiments, the selected portion of data is compressed prior to communication of the selected portion of data to the external device.

The sensing device can be implemented in a variety of ways. For example, the sensing device can communicate data to the external device when the external device telemetrically provides at least some power to the sensing device. As another example, the sensing device can discard data that substantially equals a nominal value. For yet another example, the sensing device can communicate a selected portion of data based on whether the data includes a value within a defined range of values. As still another example, the sensing device can compare data with a nominal value. For another example, the sensing device can store the selected portion of data prior to communication of the selected portion of data to the external device.

In another embodiment, a distension system for forming a distension in a patient includes an implantable distension device that can cause a distension in a patient's stomach, an implantable pressure sensing device in communication with the implantable distension device that can sense a pressure within the implantable distension device, and a processor (which can be included in the implantable pressure sensing device) that can determine whether to store any of the sensed pressure data prior to communicating any of the sensed pressure data to an external reading device. The processor, in some embodiments, can have a download of stored data to the external reading device triggered when the external reading device is moved in proximity of the implantable pressure sensing device. In some embodiments, the system can also include an external storage mechanism that can store sensed pressure data, communicate stored pressure data to an external device, and, optionally, be removably attached to the patient.

In other aspects, a method of forming a distension in a patient is provided. The method includes using an implantable pressure sensing device to obtain pressure data related to a pressure within an implantable distension device that can cause a distension in a patient, storing at least a portion of obtained pressure data at the implantable pressure sensing device, and triggering a download of stored pressure data when an external device is moved in proximity of the implantable pressure sensing device. The obtained pressure data stored at the implantable pressure sensing device can include pressure values that exceed a nominal pressure within the implantable distension device. In some embodiments, the method can also include compressing at least a portion of obtained pressure data prior to storing the at least a portion of the obtained pressure data at the implantable pressure sensing device. The compression can be performed using at least one compression technique, such as storing difference values, using a quantization table, using run-length coding, and using Huffman coding.

In another embodiment, a method of forming a distension in a patient includes obtaining pressure data related to a pressure within an implantable distension device that can cause a distension in a patient. In some embodiments, obtaining pressure data includes reducing a rate of pressure data gathering during a determined period. The method further includes determining a portion of the pressure data to retain prior to communicating pressure data to an external reading device. Determining a portion of the pressure data to retain can include determining if any of the obtained pressure data includes a value within a defined range of pressure values, determining to retain any of the obtained pressure data that exceeds a nominal pressure within the implantable distension device, and/or processing the obtained pressure data using a pressure sensing device (e.g., a processor) coupled to the implantable distension device and configured to obtain the pressure data. An alert for communication to the external reading device can be generated if any of the obtained pressure data includes a value that exceeds a threshold pressure value. In some embodiments, the method also includes storing only the portion of the pressure data determined to be retained prior to communicating pressure data to the external reading device. In still other embodiments, the method also includes compressing the portion of the pressure data determined to be retained prior to storing the portion of the pressure data determined to be retained.

In yet another embodiment, a method of forming a distension in a patient includes using an implantable pressure sensing device to obtain pressure data related to a pressure within an implantable distension device that can cause a distension in a patient, storing the obtained pressure data at the implantable pressure sensing device, and compressing the obtained pressure data prior to storing the obtained pressure data. The obtained pressure data can be compressed using at least one compression technique, such as storing difference values, using a quantization table, using run-length coding, and using Huffman coding. The method can also include communicating at least a portion of the compressed and stored pressure data from the pressure sensing device to an external device. It is understood that whereas pressure may be measured and stored, a variety of other parameters including peristaltic pulse count, pulse width, pulse amplitude, pulse duration, pH, temperature, acceleration and other relevant physiologic parameters.

The present invention generally provides devices and methods for handling data related to implantable distension devices. In general, the devices and methods allow collection, analysis, storage, and transmission of measurements related to any parameter related to implantable distension devices, such as pressure, pulse count, pulse width, and amplitude, pH, temperature, acceleration and other physiologically relevant parameters. While the methods and devices discussed herein can relate to any sensed data parameter, in an exemplary embodiment, the measurements relate to pressure. Pressure measurements can help accurately evaluate the performance of and determine any advisable pressure adjustments of an implantable distension device, but not all collected pressure data may be helpful in making such evaluations and determinations. Furthermore, handling pressure measurement data can drain power resources of an implantable distension system and can use costly, physically bulky, and electronically large data storage space. Pressure measurement data can be compressed before storing it, thereby using less storage space, time, power, and/or bandwidth for communication than for the corresponding, uncompressed data. Pressure measurement data can also be compressed prior to communication. The data can be compressed and directly transmitted, or the compressed data stored in memory can be recalled and communicated wirelessly when interrogated. Additionally, not all pressure data need be recorded or retained. Not recording or retaining all pressure data, such as data substantially equaling a resting or nominal pressure of the implantable distension device indicative of little to no pressure variation and data indicative of isolated, non-recurring events, can save storage space for potentially more analytically valuable pressure measurement data and reduce the amount of physical and/or electronic storage space used for pressure measurements. Any pressure measurement data that is recorded can be transmitted to an external device using power telemetrically provided or inductively coupled by the external device, thereby reducing or eliminating power supply resources local to the storage location of recorded data.

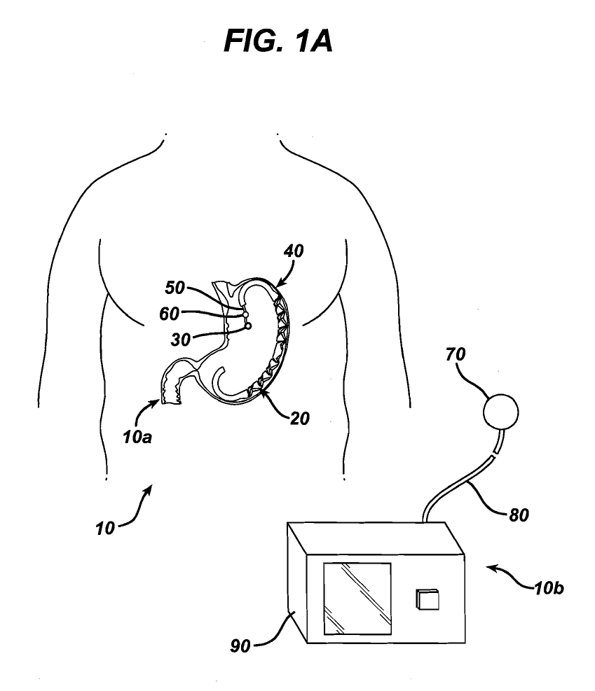

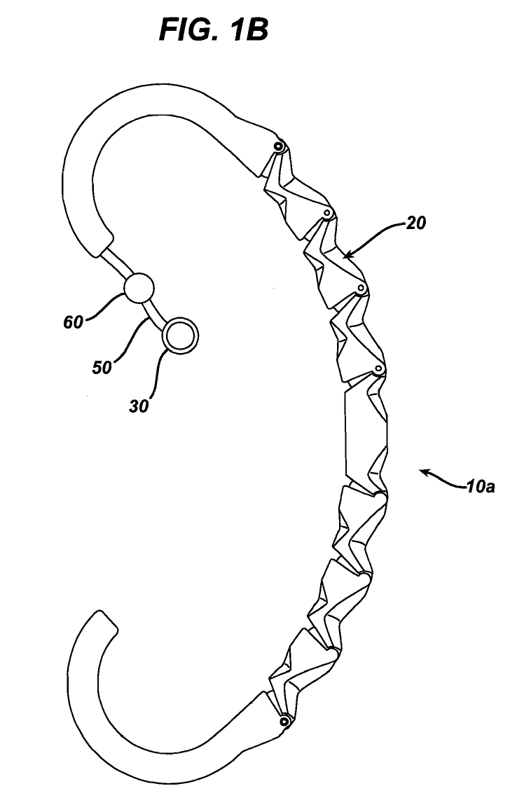

While the present invention can be used with a variety of distension systems known in the art, FIG. 1A illustrates one exemplary embodiment of a stomach distension system 10 in use in a patient. As shown, the system 10 generally includes an implantable portion 10a and an external portion 10b. FIG. 1B illustrates the implantable portion 10a outside of a patient. As shown, the implantable portion 10a includes an adjustable gastric coil 20 that is configured to be positioned in a patient's stomach 40, and an injection port housing 30 that is fluidly coupled to the adjustable gastric coil 20, e.g., via a catheter 50. The injection port 30 is adapted to allow fluid to be introduced into and removed from the gastric coil 20 to thereby adjust the size of the coil 20 and thus the pressure applied to the stomach 40. The injection port 30 can thus be implanted at a location within the body that is accessible endoscopically. Typically, injection ports are positioned on the distension device.

The internal portion 10a can also include a sensing or measuring device that is in fluid communication with the closed fluid circuit in the implantable portion 10a. In one embodiment, the sensing device is a pressure sensing device configured to measure the fluid pressure of the closed fluid circuit. While the pressure measuring device can have various configurations and it can be positioned anywhere along the internal portion 10a, including within the injection port 30 and as described further below, in the illustrated embodiment the pressure measuring device is in the form of a pressure sensor that is disposed within a sensor housing 60 positioned adjacent to the injection port 30. The catheter 50 can include a first portion that is coupled between the gastric coil 20 and the pressure sensor housing 60, and a second portion that is coupled between the pressure sensor housing 60 and the injection port 30. While it is understood that the sensing device can be configured to obtain data relating to one or more relevant parameters, generally it will be described herein in a context of a pressure sensing device.

In addition to sensing pressure of fluid within the internal portion 10a as described herein, pressure of fluid within the esophagus and/or the stomach 40 can also be sensed using any suitable device, such as an endoscopic manometer. By way of non-limiting example, such fluid pressure measurements can be compared against measured pressure of fluid within the internal portion 10a before, during, and/or after adjustment of pressure within the internal portion 10a. Other suitable uses for measured pressure within the esophagus and/or the stomach 40 will be appreciated by those skilled in the art.

As further shown in FIG. 1A, the external portion 10b generally includes a data reading device 70 that is configured to be positioned on the skin surface above the sensor housing 60 to non-invasively communicate with the sensor housing 60 and thereby obtain data (e.g., pressure) measurements. The data reading device 70 can optionally be electrically coupled (wirelessly or wired, as in this embodiment via an electrical cable assembly 80) to a control box 90 that can display the pressure measurements and/or other data obtained from the data reading device 70. While shown in this example as located local to the patient, the control box 90 can be at a location local to or remote from the patient, as explained further below.

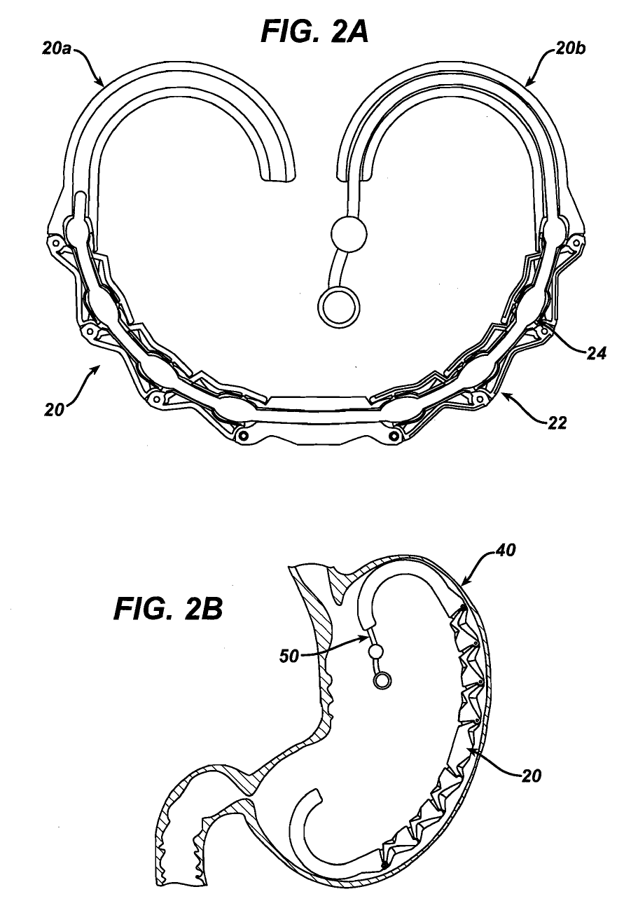

FIG. 2A shows the gastric coil 20 in more detail. While the gastric coil 20 can have a variety of configurations, and various gastric coils currently known in the art can be used with the present disclosure, in the illustrated embodiment the gastric coil 20 has a generally elongate shape with a support structure 22 having first and second opposite ends 20a, 20b that can be formed in a C-shape. Various techniques can be used to keep the ends 20a, 20b in relative proximity to one another. In the illustrated embodiment, the fluid bladder pressure may be varied to control the proximity of the ends relative to each other. The gastric coil 20 can also include a variable volume member, such as an inflatable balloon 24, that is disposed or formed on one side of the support structure 22 and that is configured to be positioned adjacent to tissue. The balloon 24 can expand or contract against the inner wall of the coil to form an adjustable size coil for controllably restricting food intake into the stomach.

A person skilled in the art will appreciate that the gastric coil can have a variety of other configurations. Moreover, the various methods and devices disclosed herein have equal applicability to other types of implantable coils.

FIG. 2B shows the adjustable gastric coil 20 applied in the stomach of a patient. As shown, the coil 20 at least substantially distends the stomach 40. After the coil 20 is implanted, it may be deployed. A person skilled in the art will appreciate that various techniques, including mechanical and electrical techniques, can be used to adjust the coil.

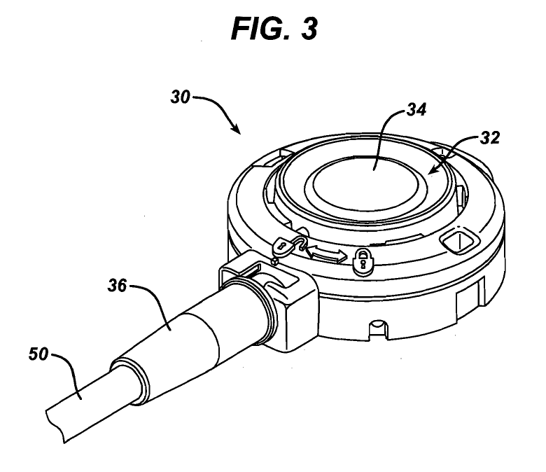

The fluid injection port 30 can also have a variety of configurations. In the embodiment shown in FIG. 3, the injection port 30 has a generally cylindrical housing with a distal or bottom surface and a perimeter wall extending proximally from the bottom surface and defining a proximal opening 32. The proximal opening 32 can include a needle-penetrable septum 34 extending there across and providing access to a fluid reservoir (not visible in FIG. 3) formed within the housing. The septum 34 is preferably placed in a proximal enough position such that the depth of the reservoir is sufficient enough to expose the open tip of a needle, such as an endoscopic Huber-like needle, so that fluid transfer can take place. The septum 34 is preferably arranged so that it will self seal after being punctured by a needle and the needle is withdrawn. As further shown in FIG. 3, the port 30 can further include a catheter tube connection member 36 that is in fluid communication with the reservoir and that is configured to couple to a catheter (e.g., the catheter 50). A person skilled in the art will appreciate that the housing can be made from any number of materials, including stainless steel, titanium, or polymeric materials, and the septum 34 can likewise be made from any number of materials, including silicone.

The reading device 70 can also have a variety of configurations, and one exemplary pressure reading device is disclosed in more detail in commonly-owned U.S. Publication No. 2006/0189888 and U.S. Publication No. 2006/0199997, which are hereby incorporated by reference. In general, the data reading device 70 can non-invasively measure the pressure of the fluid within the implanted portion 10a even when the pressure sensing device is implanted beneath thick (at least over 10 cm) subcutaneous fat tissue in the patient's stomach. The physician can hold the reading device 70 against the patient's skin near the location of the sensor housing 60, and/or other pressure sensing device location(s), and observe the pressure reading on a display on the control box 90. The data reading device 70 can also be removably attached to the patient, as discussed further below, such as during a prolonged examination, using straps, adhesives, and other well-known methods. The data reading device 70 can operate through conventional cloth or paper surgical drapes, and can also include a disposal cover (not shown) that may be replaced for each patient. Furthermore, the reading device may be operated using an endoscopic probe which may be inserted down the mouth of the patient to close proximity with the coil.

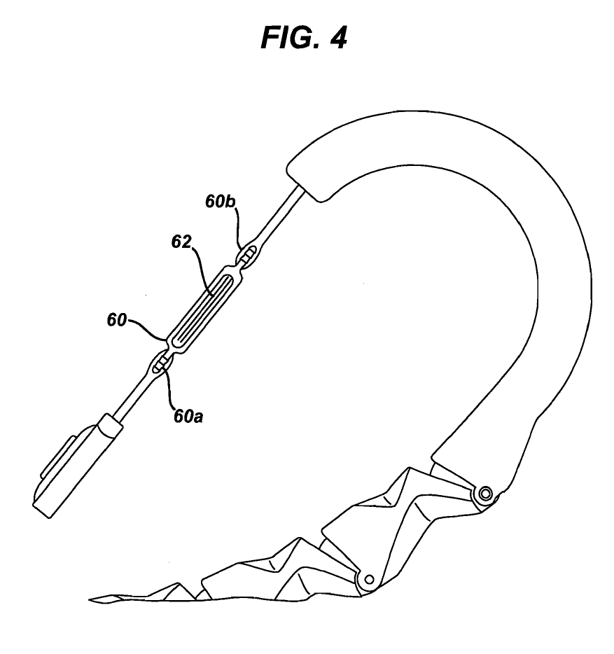

As indicated above, the system 10 can also include a pressure measuring device in communication with the closed fluid circuit and configured to measure pressure (e.g., fluid pressure) which corresponds to the amount of distension applied by the adjustable gastric coil 20 to the patient's stomach 40. Measuring the pressure enables a person (e.g., a physician, a nurse, a patient, etc.) to evaluate the efficacy and functionality of the distension created by a coil adjustment. In the illustrated embodiment, as shown in FIG. 4, the pressure measuring device is in the form of a pressure sensor 62 disposed within the sensor housing 60. The pressure measuring device can, however, be disposed anywhere within the closed hydraulic circuit of the implantable portion, and various exemplary locations and configurations are disclosed in more detail in commonly-owned U.S. Publication No. 2006/0211913 entitled “Non-Invasive Pressure Measurement In a Fluid Adjustable Restrictive Device,” filed on Mar. 7, 2006, and hereby incorporated by reference. In general, the illustrated sensor housing 60 includes an inlet 60a and an outlet 60b that are in fluid communication with the fluid in the implantable portion 10a. An already-implanted catheter 50 can be retrofitted with the sensor housing 60, such as by severing the catheter 50 and inserting barbed connectors (or any other connectors, such as clamps, clips, adhesives, welding, etc.) into the severed ends of the catheter 50. The sensor 62 can be disposed within the housing 60 and be configured to respond to fluid pressure changes within the hydraulic circuit and convert the pressure changes into a usable form of data.

Various pressure sensors known in the art can be used as the pressure sensor 62, such as a wireless pressure sensor provided by CardioMEMS, Inc. of Atlanta, Ga., though a suitable MEMS pressure sensor may be obtained from any other source, including but not limited to Integrated Sensing Systems, Inc. (ISSYS) of Ypsilanti, Mich. and Remon Medical Technologies, Inc. of Waltham, Mass. One exemplary MEMS pressure sensor is described in U.S. Pat. No. 6,855,115, the disclosure of which is incorporated by reference herein for illustrative purposes only. It will also be appreciated by a person skilled in the art that suitable pressure sensors can include, but are not limited to, capacitive, piezoresistive, silicon strain gauge, or ultrasonic (acoustic) pressure sensors, as well as various other devices capable of measuring pressure.

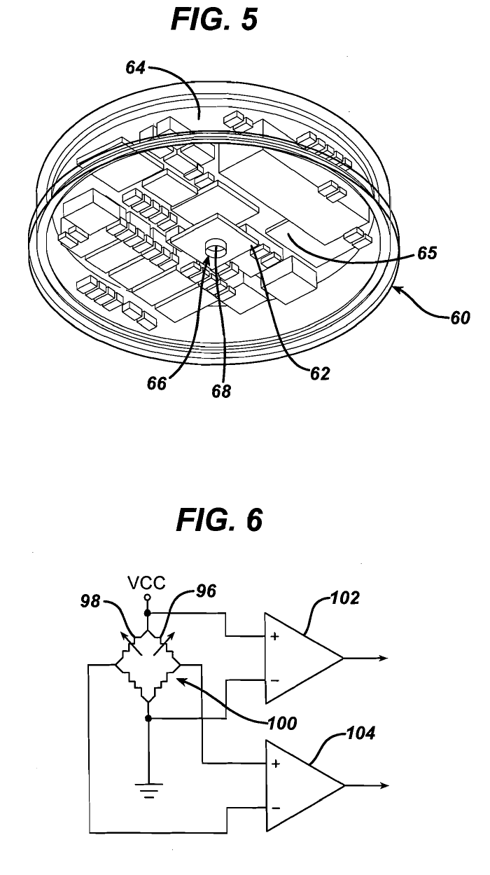

One embodiment of a configuration of the sensor housing 60 having the sensor 62 disposed within it is shown in FIG. 5. The sensor housing 60 in this example includes a motherboard that can serve as a hermetic container to prevent fluid from contacting any elements disposed within the sensor housing 60, except as discussed for the sensor 62. The sensor housing 60 can be made from any biocompatible material appropriate for use in a body, such as a polymer, biocompatible metal, and other similar types of material. Furthermore, the sensor housing 60 can be made from any one or more of transparent (as shown in FIG. 5), opaque, semi-opaque, and radio-opaque materials. A circuit board 64 including, among other elements, a microcontroller 65 (e.g., a processor), can also be disposed within the housing 60 to help process and communicate pressure measurements gathered by the sensor 62, and also possibly other data related to the coil 20. As further discussed below, the circuit board 64 can also include a transcutaneous energy transfer (TET)/telemetry coil and a capacitor. Optionally, a temperature sensor can be integrated into the circuit board 64. The microcontroller 65, the TET/telemetry coil, the capacitor, and/or the temperature sensor can be in communication via the circuit board 64 or via any other suitable component(s). The TET/telemetry coil and capacitor can collectively form a tuned tank circuit for receiving power from the external portion 10b, and transmitting pressure measurements to a pressure reading device, e.g., the reading device 70. Moreover, to the extent that a telemetry component associated with the pressure sensor 62 is unable to reach a telemetry device external to the patient without some assistance, such assistance can be provided by any suitable number of relays (not shown) or other devices.

Fluid can enter the sensor housing 60 through an opening 66 located anywhere on the housing's surface (here, its bottom surface) and come into contact with a pressure sensing surface 68 of the sensor 62. The sensor 62 is typically hermetically sealed to the motherboard such that fluid entering the opening 66 cannot infiltrate and affect operation of the sensor 62 except at the pressure sensing surface 68. The sensor 62 can measure the pressure of fluid coming into contact with the pressure sensing surface 68 as fluid flows in and out of the opening 66. For example, the pressure sensing surface 68 can include a diaphragm having a deformable surface such that when fluid flows through the opening 66, the fluid impacts the surface of the diaphragm, causing the surface to mechanically displace. The mechanical displacement of the diaphragm can be converted to an electrical signal by a variable resistance circuit including a pair of variable resistance, silicon strain gauges. One strain gauge can be attached to a center portion of diaphragm to measure the displacement of the diaphragm, while the second, matched strain gauge can be attached near the outer edge of diaphragm. The strain gauges can be attached to the diaphragm with adhesives or can be diffused into the diaphragm structure. As fluid pressure within the gastric coil 20 fluctuates, the surface of the diaphragm can deform up or down, thereby producing a resistance change in the center strain gauge.

One embodiment of a variable resistance circuit for the sensor 62 is shown in FIG. 6. The circuit includes first and second strain gauges 96, 98 that form the top two resistance elements of a half-compensated, Wheatstone bridge circuit 100. As the first strain gauge 96 reacts to the mechanical displacements of the sensor's diaphragm, the changing resistance of the first gauge 96 changes the potential across the top portion of the bridge circuit 100. The second strain gauge 98 is matched to the first strain gauge 96 and athermalizes the Wheatstone bridge circuit 100. First and second differential amplifiers 102, 104 are connected to the bridge circuit 100 to measure the change in potential within the bridge circuit 100 due to the variable resistance strain gauges 96, 98. In particular, the first differential amplifier 102 measures the voltage across the entire bridge circuit 100, while the second differential amplifier 104 measures the differential voltage across the strain gauge half of bridge circuit 100. The greater the differential between the strain gauge voltages, for a fixed voltage across the bridge, the greater the pressure difference. Output signals from the differential amplifiers 102, 104 can be applied to the microcontroller 65 integrated into the circuit board 64, and the microcontroller 65 can transmit the measured pressure data to a device external to the patient. If desired, a fully compensated Wheatstone bridge circuit can also be used to increase the sensitivity and accuracy of the pressure sensor 62. In a fully compensated bridge circuit, four strain gauges are attached to the surface of diaphragm rather than only two strain gauges.

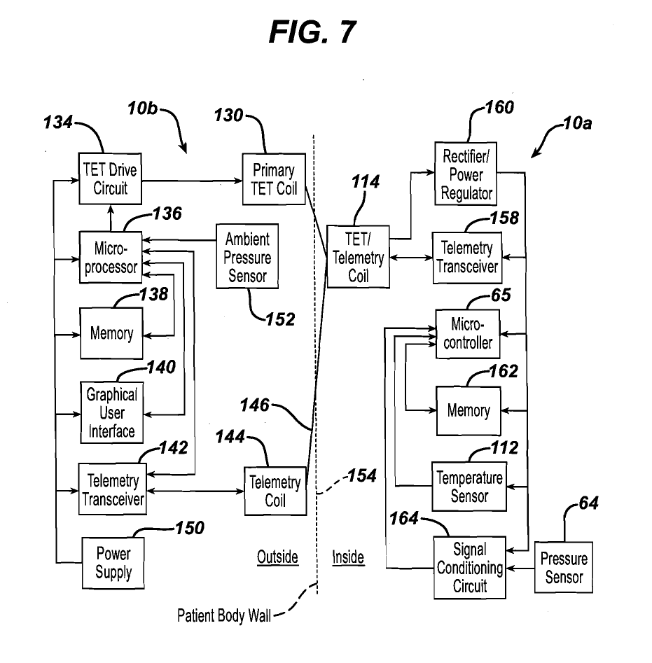

FIG. 7 illustrates one embodiment of components included in the internal and external portions 10a, 10b of the stomach distension system 10. As shown in FIG. 7, the external portion 10b includes a primary TET coil 130 for transmitting a power signal 132 to the internal portion 10a. A telemetry coil 144 is also included for transmitting data signals to the internal portion 10a. The primary TET coil 130 and the telemetry coil 144 combine to form an antenna, e.g., the reading device 70. The external portion 10b, e.g., the control box 90, includes a TET drive circuit 134 for controlling the application of power to the primary TET coil 130. The TET drive circuit 134 is controlled by a microprocessor 136 having an associated memory 138. A graphical user interface 140 is connected to the microprocessor 136 for inputting patient information and displaying and/or printing data and physician instructions. Through the user interface 140, a user such as the patient or a clinician can transmit an adjustment request to the physician and also enter reasons for the request. Additionally, the user interface 140 can enable the patient to read and respond to instructions from the physician and/or pressure measurement alerts, as discussed further below.

The external portion 10b also includes a primary telemetry transceiver 142 for transmitting interrogation commands to and receiving response data, including sensed pressure data, from the implanted microcontroller 65. The primary transceiver 142 is electrically connected to the microprocessor 136 for inputting and receiving command and data signals. The primary transceiver 142 drives the telemetry coil 144 to resonate at a selected RF communication frequency. The resonating circuit can generate a downlink alternating magnetic field 146 that transmits command data to the microcontroller 65. Alternatively, the transceiver 142 can receive telemetry signals transmitted from a secondary TET/telemetry coil 114 in the internal portion 10a. The received data can be stored in the memory 138 associated with the microprocessor 136. A power supply 150 can supply energy to the control box 90 in order to power element(s) in the internal portion 10a. An ambient pressure sensor 152 is connected to microprocessor 136. The microprocessor 136 can use a signal from the ambient pressure sensor 152 to adjust the received pressure measurements for variations in atmospheric pressure due to, for example, variations in barometric conditions or altitude, in order to increase the accuracy of pressure measurements.

FIG. 7 also illustrates components of the internal portion 10a, which in this embodiment are included in the sensor housing 60 (e.g., on the circuit board 64). As shown in FIG. 7, the secondary TET/telemetry coil 114 receives the power/communication signal 132 from the external antenna. The secondary coil 114 forms a tuned tank circuit that is inductively coupled with either the primary TET coil 130 to power the implant or the primary telemetry coil 144 to receive and transmit data. A telemetry transceiver 158 controls data exchange with the secondary coil 114. Additionally, the internal portion 10a includes a rectifier/power regulator 160, the microcontroller 65, a memory 162 associated with the microcontroller 65, a temperature sensor 112, the pressure sensor 62, and a signal conditioning circuit 164. The implanted components can transmit pressure measurements (with or without adjustments due to temperature, etc.) from the sensor 62 to the control box 90 via the antenna (the primary TET coil 130 and the telemetry coil 144). Pressure measurements can be stored in the memory 138, adjusted for ambient pressure, shown on a display on the control box 90, and/or transmitted, possibly in real time, to a remote monitoring station at a location remote from the patient.

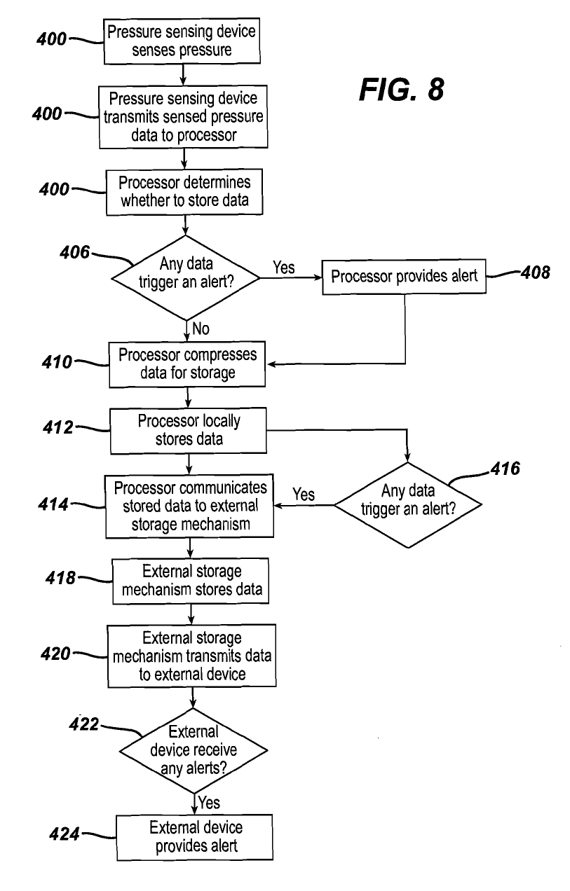

As illustrated in a process shown in FIG. 8, the sensor housing 60 can generally sense pressure within the gastric coil 20, locally store the sensed pressure data (e.g., at the memory 162), and communicate at least a portion of the sensed pressure data to an external device such as the control box 90 via the reading device 70. While the pressure sensor 62 can communicate all pressure data it senses to the reading device 70, communicating only a selected portion of the pressure data (e.g., a portion less than the entirety of the sensed pressure data) can use less power, require less physical and/or electronic storage space in the sensor housing 60, and/or reduce costs.

While the process shown in FIG. 8 is discussed with relation to the elements included in FIGS. 1A-7, a person skilled in the art will appreciate that the process can be modified to include more or fewer elements, reorganized or not, and can be performed in the system 10 or in another, similar system having other, similar elements. For example, the microcontroller 65 processes instructions in this embodiment, but any processor configured to process instructions for a system (e.g., a central processing unit, a microprocessor, a digital signal processing unit, application specific integrated circuits (ASICs), a state machine, an analog computer, an optical or photonic computer, logic circuitry, etc.) can be used. Furthermore, the sensor 62 in this illustrated embodiment measures fluid pressure, but any sensed pressure data related to the coil 20 can be handled as discussed herein.

In use, the sensor housing 60 can sense 400 a pressure of fluid disposed within the system 20 using the sensor 62. The sensor 62 can transmit measured signals to the signal conditioning circuit 164, which can amplify the signals before the signal conditioning circuit 164 transmits 402 the measured pressure data to the microcontroller 65. Alternatively, in some embodiments, the sensor 62 can directly transmit signals to the microcontroller 65. In this embodiment, the pressure sensor 62 provides pressure data at an update rate of approximately 20 Hz. Such a rate can provide a telemetry/TET mode cycle completion at approximately every 50 ms. For example, the TET/telemetry coil 114 can provide TET for the sensor housing 60 for approximately 45 ms to power the sensor housing 60 and then provide telemetry of pressure data for approximately 5 ms. Of course, any other switching topology can be used. It will also be appreciated that switching between TET and telemetry may be unnecessary. For example, the sensor housing 60 can be active, such that TET is not required. As another example, a second coil (not shown) can be added to the sensor housing 60, with one of the coils in the sensor housing 60 being dedicated to TET and the other to telemetry. Still other alternatives and variations will be apparent to those of ordinary skill in the art.

Having received sensed pressure data, the microcontroller 65 can determine 404 whether to store 412 the data, e.g., in the memory 162. Any type of memory can be used for the memory 162, including but not limited to one or more of volatile (e.g., SRAM, etc.), non-volatile (e.g., flash, hard drive, etc.), or other memory. Determining whether to store the data allows the microcontroller 65 to analyze the data and potentially discard at least a portion of the data before storing it, thereby using less of the storage space available in the memory 162. The microcontroller 65 can, however, be configured to store 412 all sensed pressure data and thus may not make such a determination and instead proceed to evaluating 406 whether any of the data triggers an alert, as further discussed below. (In such a configuration, it may be more power efficient to store raw (unprocessed) data from the pressure sensor 62 and process the raw data via an external reading device.) Furthermore, the memory 162 can be used to store pre-selected information or pre-selected types of information. For example, the memory 162 can store maximum, minimum, and/or baseline, pressure measurements, pressure profiles, pressure trends, and/or any other information. Other information suitable for storing in the memory 162 will be appreciated by those skilled in the art.

The microcontroller 65 can analyze the data in a variety of ways in determining whether to store it. Typically, the microcontroller 65 analyzes a sequence of pressure data values measured over a period of time rather than analyzing every discrete pressure measurement, thereby allowing analysis of pressure trends over time and saving processing resources by not necessarily having to continually analyze incoming data. The microcontroller 65 can, however, evaluate individual pressure data measurements (and/or a range of data) for invalid data and determine to discard any invalid data. Generally, in determining whether to store data, the microcontroller 65 considers a variation of pressure data from a nominal pressure, or resting pressure, within the coil 20. The nominal pressure is typically programmed into the microcontroller 65 by a physician based on historical coil performance in the patient or, particularly for recently implanted coils, in a typical patient. If the measured pressure data exceeds the nominal pressure, then the data indicates pressure variation in the system 10 and hence likely includes potentially beneficial information for analytical, diagnostic, and/or other purposes. If the pressure data substantially equals the nominal pressure, then the data is not likely indicative of a potentially significant event for analysis purposes, e.g., a change in coil pressure due to patient activity such as eating or drinking. The microcontroller 65 can discard any such substantially nominal data. Discarding data can include not storing the data or storing a representation of the data, e.g., storing a specific set of digits (e.g., “888,” “999,” “000,” etc.) or one or more alphabetic characters. Different representations of data can be used to indicate measurement of a different types of data, e.g., substantially nominal data, data outside a defined pressure range, etc. Although, in some embodiments, the microcontroller 65 can store 412 even nominal pressure data in the memory 162 to maintain a complete historical record of pressure measurements. Furthermore, the microcontroller 65 can store 412 all sensed pressure data it receives in the memory 162 and subsequently determine whether to keep or discard it, e.g., store all data and analyze it every “X” minutes and/or upon signal from an external device.

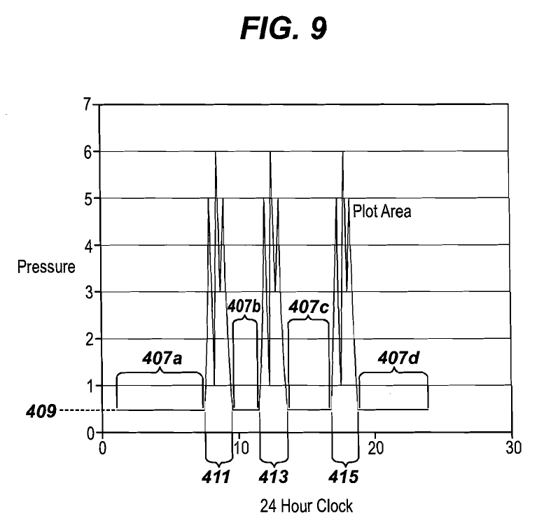

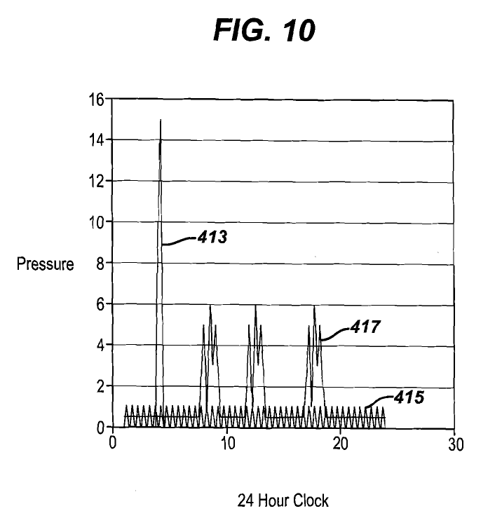

FIGS. 9 and 10 show example sequences of pressure data that the microcontroller 65 can receive from the sensor 62. In each of FIGS. 9 and 10, a plot shows sensed pressure data versus time for a twenty-four hour period. The plot in FIG. 9 includes four periods 407a, 407b, 407c, 407d of substantially nominal pressure at a nominal pressure level 409. The nominal pressure level 409 shown in the plot is an example only; the nominal pressure value can be any value or range of values. Furthermore, the nominal pressure value for a patient can change over time, e.g., as the patient loses weight. The microcontroller 65 can compare the pressure data from this twenty-four hour period with the nominal pressure 409 and determine to discard data from the nominal pressure periods 407a, 407b, 407c, 407d (e.g., never store it in the memory 162 or delete it from the memory 162) and only store 412 the remaining, selected portion of pressure data. In some instances, the microcontroller 65 can determine to discard pressure data that exceeds the nominal pressure 409. For example, the microcontroller 65 can discard pressure data except for data obtained during two of three meals the patient ate during the day, e.g., discard pressure data measured during the four periods 407a, 407b, 407c, 407d and during a breakfast period 411 and store 412 the remaining, selected pressure data, corresponding to lunch and dinner periods 413, 415. Pressure data can be determined to be related to a particular meal based on one or more factors considered by the microcontroller 65, such as a combination of a time of day when the sensor 62 measured the data and a duration of pressure values above the nominal level 409.

The microcontroller 65 can also determine to discard pressure data related to one or more physiologic events, as illustrated in FIG. 10. Non-limiting examples of physiologic events include supra events (e.g., coughing, vomiting, wretching, etc.) and normal events (heartbeats, breathing, talking, etc.). Physiologic events can result in measured pressure data that significantly differs from an expected level in magnitude, duration, occurrence (e.g., an unexpected time of day, such as midnight), and/or frequency from established patterns of patient eating. The microcontroller 65 can determine to retain pressure data by analyzing the data for such a significant difference, such as by determining if any of the obtained pressure data includes a value above a pre-programmed threshold value typically not exceeded except in response to a physiologic event. The microcontroller 65 can also or instead determine if any of the obtained pressure data includes a value within a defined range of pressure values. Depending on the defined range, which can in some embodiments be defined at an upper and/or lower limit by an immediately preceding pressure data value or by pressure values corresponding to a particular time of day, the microcontroller 65 can determine to discard data within the range (e.g., if the range reflects pressure readings of an expected frequency and magnitude caused by a normal event) or to retain data within the range (e.g., if the range includes any positive pressure values up to a threshold value typically not exceeded except by a physiologic event). As an example, the plot in FIG. 10 includes pressure data 413 indicative of a super-physiologic event, pressure data 415 indicative of a normal event, and actual gastric coil pressure data 417. The microcontroller 65 can discard the super-physiologic event data 413 and the normal event data 415 using one or more programmed algorithms as described above.

The microcontroller 65 can also determine 406 whether any data triggers an alert. If the microcontroller 65 determines that any pressure data falls outside a defined range of pressure values and/or is more or less than a threshold value, then the microcontroller 65 can provide 408 an alert to a physician, the patient, and/or to any number of other people because such outlying pressure data can indicate a possible problem such as coil bladder leakage, coil over-extension, recurrent wretching, coil slippage, erosion, etc. The microcontroller 65 can provide the alert by, for example, communicating a signal to an external device (e.g., the control box 90) indicating the potentially problematic sensed pressure data and triggering notice of the alert. An alert can include any one or more of the following: an e-mail, a phone call, a text message, an audible signal, a mechanical vibration, a light or other visual display, a tactile display, a message displayed on an external device, or any other type of alert. Different alert patterns (e.g., varying audio signals, varying vibration patterns, etc.) can be used to signify different conditions. Two or more alerts can be provided to multiple people under similar conditions, although alerts may not be provided simultaneously to multiple people or be provided to anyone at all. The conditions for and/or the type of an alert can also vary relative to the recipient of the alert. For example, with respect to alerts for physicians or other medical personnel, such alerts may be limited to those provided upon a super-event indicating that some component of the internal portion 10a has structurally failed (e.g., a kink in catheter 50, a leak in the coil bladder 24, etc.). With respect to alerts for patients, such alerts may be limited to patient activity such as those provided upon an indication that the patient is eating too much, or eating too quickly. A variety of other conditions under which alerts can be directed to a physician, a patient, and/or another person will be understood by those skilled in the art. Other suitable processes for detecting alert triggers, as well as ways in which the alerts can be provided and the timing of providing the alerts (e.g., immediately, on a regular schedule such as every day or every hour, after detection of a certain milestone or pattern of data, etc.), will be appreciated by those skilled in the art.

The microcontroller 65 can optionally compress 410 data prior to storing 412 data in the memory 162. Such compression can reduce the amount of memory space required to store data in the internal portion 10a (and subsequently in the external portion 10b), reduce the number of microcontroller accesses to the memory 162 (thereby saving power), reduce the amount of time and/or power required to communicate data from the sensor housing 60 to an external device, and allow more data to be locally stored prior to communicating the data to an external device. While pressure data is shown in FIG. 8 as being compressed following a determination of a selected portion of data to store in the memory 162, if any, the microcontroller 65 can compress data before making such as determination. For example, as mentioned above, the microcontroller 65 can store 412 pressure data prior to making such a determination (possibly subsequently retrieving the data for analysis). As another example, the microcontroller 65 may not be configured to perform such determining analysis and may store 412 all data for communication to an external device.

The microcontroller 65 can compress data using any one or more lossless and/or lossy compression techniques. Non-limiting examples of lossless compression techniques include recording difference values (instead of absolute values), reducing the sensor's data sampling rate (which can include reducing the sensor's data sampling rate to zero) during a determined period (e.g., a period of quiescent pressure, after a certain period of data-gathering time, etc.), run-length coding, Huffman coding, and other types of lossless compression. Non-limiting examples of lossy compression includes using a quantization table (e.g., sparse quantization) and other types of lossy compression. Storing difference values instead of absolute values can be effective compression if, typically at the beginning of pressure measuring and at regular intervals, the microcontroller 65 stores an absolute value in the memory 162 that can serve as a baseline in reconstructing the originally sensed data. Sensed pressure values are often near the values of their neighbors, so differences from a baseline are often likely to be small, if not zero. The microcontroller 65 can compress difference values for storage using a compression technique, such as encoding difference values into the shortest code symbols in Huffman coding.

Data stored in the memory 162 can be communicated 414 to an external device. In some embodiments, the microcontroller 65 continually communicates 414 data (via the telemetry transceiver 158 and the secondary coil 114), and the data is only received when an appropriate receiving device, such as the antenna (the primary TET coil 130 and the telemetry coil 144), moves into sufficient proximity of it. In some embodiments, a download of data from the memory 162 can be triggered 416 when an external device (e.g., the reading device 70) telemetrically provides power to the sensor housing, e.g., when the external device is moved in proximity of the sensor housing 60. The external device can be mobile (e.g., a wand or hand-held unit that can be waved or otherwise placed in proximity of the sensor housing 60) or stationary (e.g., a bedside, desk-mounted, or car-mounted box that the patient can move near). Telemetrically providing power to the sensor housing 60 can save power in the internal portion 10a because download communication power is supplied by the external portion 10b.

The external device can be configured to store 418 data received from the sensor housing 60. The external device can be further configured communicate 420 the data to another external device, such as a base unit at a location remote from the patient. The external device (typically, the control box 90 or other device having a capability to display or otherwise provide an alert) can detect 422 if the internal portion 10a communicated a signal indicating an alert and provide 424 an alert as appropriate (e.g., displaying a warning notice, sending an e-mail message, etc.).

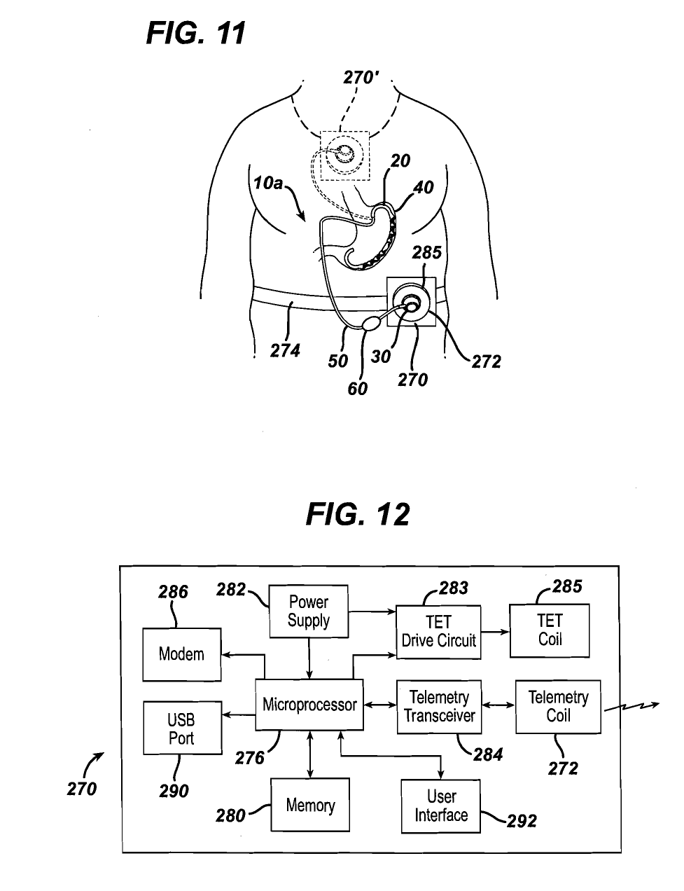

As mentioned above, a pressure history (e.g., pressure data gathered by the sensor 62) can be uploaded to the control box 90 (and/or other units located local or remote to the patient) to allow a person to physically evaluate and/or the control box 90 to electronically evaluate the patient's treatment and/or performance of elements included in the internal portion 10a over a designated time period. FIG. 11 illustrates an embodiment of an external device, a data logger 270, that can be used as an external storage mechanism to store pressure measurements over a period of time. The data logger 270 can function as a removably attached data reading device 70, mentioned above. In this example, the data logger 270 includes a wearable pack external to the patient worn on a belt 274 and positioned over or within communication range of the region under which the sensor housing 60 is implanted within the patient. Alternatively, the data logger 270 can be worn about the patient's neck, as shown by a device 270′, such as when the injection port 30 is implanted in the patient's stomach and the port 30 includes the pressure sensing device. In another embodiment, the data logger 270 is also implanted within the patient.

As shown in FIG. 11, the data logger 270 includes a TET coil 285 and a telemetry coil 272 which can be worn by the patient so as to lie adjacent to the internal portion 10a. The TET coil 285 can provide power to the implant, while the telemetry coil 272 can interrogate the implant and can receive data signals, including pressure measurements, through the secondary telemetry coil 114 in the implanted portion 10a. In another embodiment, the TET coil 285 and the telemetry coil 272 can be consolidated into a single coil and alternate between TET and telemetry functions at any suitable rate for any suitable durations.

The pressure within the coil 20 can be repeatedly sensed and transmitted to the data logger 270 at an update rate sufficient to measure peristaltic pulses against the coil 20. Typically, this update rate is in the range of 10-20 pressure measurements per second, but any update range can be used. The data logger 270 is typically worn during waking periods to record pressure variations during the patient's meals and daily routines. At the end of the day, or another set time period, the data logger 270 can be removed and recorded pressure data downloaded to the external memory 138. The pressure history can be uploaded from the memory 138 to a remote unit over one or more communication links during a subsequent communication session. Alternatively, pressure data can be directly uploaded from the data logger 270 to a remote unit using one or more communication links. A communication link can include any single or combination of two or more data transmission media including web-based systems utilizing high-speed cable or dial-up connections, public telephone lines, wireless RF networks, Bluetooth, ultrawideband (UWB), satellite, T1 lines or any other type of communication media suitable for transmitting data between remote locations. The data logger 270 can be configured to dock into another device, e.g., a docking station, that is configured to receive data communication from the data logger 270 and transmit the received data to a remote unit.

FIG. 12 shows the data logger 270 in greater detail. As shown in FIG. 12, the data logger 270 includes a microprocessor 276 for controlling telemetry communications with the internal portion 10a. The microprocessor 276 is connected to a memory 280 for, at least, storing pressure measurements from the internal portion 10a. In this embodiment, the memory 280 includes forty MB of Non-Volatile EEPROM or FLASH memory and is configured to store about one hundred hours of time stamped pressure data, but any other type of storage can be used, and the memory 280 can store any amount of and any type of data. By way of non-limiting example, any other type of volatile memory or any type of non-volatile memory can be used, including but not limited to flash memory, hard drive memory, etc. While the data logger 270 in this example is operational, pressure can be read and stored in the memory 280 at a designated data rate controlled by the microprocessor 276.

The microprocessor 276 can be energized by a power supply 282. In one embodiment, the power supply 282 includes a rechargeable cell (not shown), such as a rechargeable battery. In some embodiments, the rechargeable cell is removable and can be recharged using a recharging unit and replaced with another rechargeable cell while the spent cell is recharging. In other embodiments, the rechargeable cell can be recharged by plugging a recharging adapter into the data logger 270 and a wall unit. In yet another embodiment, the rechargeable cell can be recharged wirelessly by a wireless recharging unit. In still another embodiment, the power supply 282 includes an ultra capacitor, which can also be recharged. Of course, any other type of power supply can be used.

To record pressure, the microprocessor 276 can initially transmit a power signal to the internal portion 10a via a TET drive circuit 283 and the TET coil 285. After transmitting the power signal, the microprocessor 276 can transmit an interrogation signal to the internal portion 10a via a telemetry transceiver 284 and the telemetry coil 272. The interrogation signal can be intercepted by the telemetry coil 114 and transmitted to the microcontroller 65. The microcontroller 65 can send a responsive, optionally-temperature-adjusted pressure reading from the sensor 62 via the transceiver 158 and the secondary telemetry coil 114. The pressure reading can be received through the telemetry coil 272 and directed by the transceiver 284 to the microprocessor 276. The microprocessor 276 can store the pressure measurement and initiate the next interrogation request. If applicable, the microprocessor 276 can also respond to an alert identified by the microcontroller 65, such as with a visual alert (e.g., flashing a light on the data logger 270, displaying a message on a user interface 292, etc.) and/or with an audible alert. The user interface 292 can include any number and types of features, including but not limited to a speaker, an LED, an LCD display, an on/off switch, etc. In some embodiments, the user interface 292 is configured to provide only output to the patient and does not permit the patient to provide input to the data logger 270. The user interface 292 thus includes an LED, which when lit shows that the power supply 282 is sufficiently charged and another, differently colored LED to show when the power supply 282 needs to be recharged, although such power indicators can be shown using any type and any combination of indicators such as one light that illuminates upon low power charge, an audible alert, an email alert, etc. In other embodiments, the user interface 292 can allow the patient to provide input to the data logger 270 and can accordingly include any suitable components and features.

When finished measuring and recording pressure, the data logger 270 can be removed from the patient and/or from the belt 274 and the recorded pressure data downloaded to the control box 90 (and/or to any other external device). The data logger 270 can include a modem 286 for transmitting sensed pressure data directly to a remote base unit using a communication link. For example, the patient can connect the modem 286 to a telephone line (or other communication link), dial the physician's modem (if necessary), and select a “send” button on the user interface 292. Once connected, the microprocessor 276 can transmit stored pressure history through the phone line to a microprocessor included in the remote unit. Alternatively, the data logger 270 can include a USB port 290 for connecting the logger 270 to the control box 90. The logger USB port 290 can be connected to a USB port included on the control box 90 and the “send” switch activated to download pressure data to the memory 138 in the control box 90. After pressure data is downloaded, the logger 270 can be turned off through the user interface 292 or reset and placed back on the patient and/or the belt 274 for continued pressure measurement.

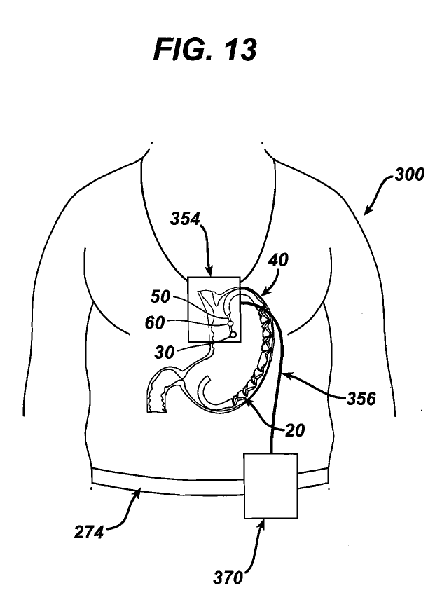

An alternate embodiment of a data logging system 300 is shown in FIG. 13. In this example, the data logging system 300 includes a coil head 354 and a data logger 370. The coil head 354 and the data logger 370 are in communication via a detachable cable 356. Any one or more suitable alternative communication links can be used in the place of the cable 356, including but not limited to a wireless transmitter/receiver system. In the illustrated embodiment, the coil head 354 is worn around the neck of the patient and is positioned generally over the injection port 30 and within communication range of the sensor housing 60. The data logger 370 is worn on the belt 274 about the patient's waist. Of course, these respective locations are merely exemplary, and either or both the coil head 354 and the data logger 370 can be positioned elsewhere. By way of non-limiting example, when the injection port 30 is implanted in the patient's abdomen, the coil head 354 can be worn on the belt 274. The coil head 354 and the data logger 370 are represented as simple blocks in FIG. 13 for illustrative purposes only, and either of the coil head 354 or the data logger 370 can be provided in a variety of shapes, sizes, and configurations.

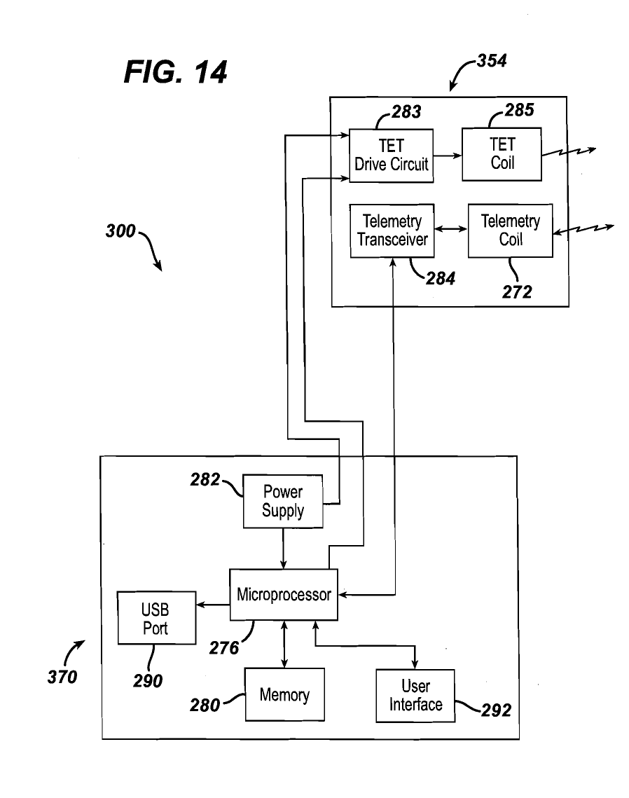

Exemplary components of the data logging system 300 are shown in FIG. 14. As shown, the data logger 370 includes the microprocessor 276, the memory 280, the power supply 282, the USB port 290, and the user interface 292. The coil head 354 includes the TET drive circuit 283, the telemetry transceiver 284, the TET coil 285, and the telemetry coil 272. The TET drive circuit 283 is configured to receive power from the power supply 282 via the cable 356. The TET drive circuit 283 is further configured to receive signals from the microprocessor 276 via the cable 356. The telemetry transceiver 284 is configured to receive signals from the microprocessor 276 and transmit signals to the microprocessor 276, via the cable 356. In another embodiment, the telemetry transceiver 284 is configured to only transmit signals to the microprocessor 276. The above discussion of such components with reference to FIG. 12 can also be applied to the components shown in FIG. 14. In the embodiment illustrated in FIG. 14, the coil head 354 and the data logger 370 can be viewed as a separation of components including the data logger 270 (described above) into two physically separate units. It will be appreciated by a person skilled in the art that any of the components shown in FIG. 14, as well as their relationships, functions, etc., can be varied in any suitable way.

In the present example, the coil head 354 is configured similar to and functions in a manner similar to the antenna (the primary TET coil 130 and the telemetry coil 144) described above. The TET coil 285 of coil head 354 is configured to provide power to the injection port 30. Of course, to the extent that any other devices (e.g., a pump, etc.) are implanted in the patient that are configured to receive power from the TET coil 285, the TET coil 285 can also provide power to such devices. Power provided by the TET coil 285 can be provided to the TET coil 285 by and regulated by the TET drive circuit 285, which can itself receive power from the power supply 282 via the cable 356. Such power provided to the TET drive circuit 283 can be regulated by the microprocessor 276 via the cable 356. In addition, or in the alternative, the microprocessor 276 can regulate the manner in which the TET drive circuit 285 provides power to the TET coil 285. While the present example contemplates the use of RF signaling through the TET coil 285, any other type of powering technique, as well as alternative power communicators, can be used. Other suitable configurations and relationships between these components, as well as alternative ways in which they may operate, will be appreciated by those skilled in the art.

The telemetry coil 272 of the coil head 354 is configured to receive signals from the coil 114, including signals indicative of the pressure within the implanted coil system (e.g., pressure of fluid within the injection port 30, within the catheter 50, and/or within the adjustable coil 20, pressure obtained using the pressure sensor 62, etc.) and signals indicative of temperature. The telemetry coil 272 can also receive any other type of signal representing any other type of information from any other source. Signals received by the telemetry coil 272 can be communicated to the telemetry transceiver 284, which can communicate such signals to the microprocessor 276 via the cable 356. The telemetry transceiver 284 can perform any appropriate translation or processing of signals received from the telemetry coil 272 before communicating signals to the microprocessor 276. Other suitable configurations and relationships between these components, as well as alternative ways in which they may operate, will be appreciated by those skilled in the art. It will also be appreciated that components may be combined. By way of non-limiting example, the TET coil 285 and the telemetry coil 272 can be consolidated into a single coil and alternate between TET and telemetry functions at any suitable rate for any suitable durations. In addition, while the present example contemplates the use of RF signaling through the telemetry coil 272, it will be appreciated that any other type of communication technique (e.g., ultrasonic, magnetic, RF, light, inductive, etc.) can be used alone or in any combination, as well as alternative communicators other than a coil, can be used. Furthermore, different data handling can be more beneficial to a given communication technique, and given a particular communication technique, appropriate data handling can be selected.

In one exemplary use, the patient wears the coil head 354 and the data logger 370 throughout the day to record pressure measurements in the memory 280. At night, the patient can decouple the data logger 370 from the coil head 354 and couple the data logger 370 with a docking station, e.g., the control box 90. While the data logger 370 and the control box 90 are coupled, the control box 90 can transmit data received from the data logger 370 to a remote unit. To the extent that the power supply 282 includes a rechargeable cell, the control box 90 can recharge the cell while the data logger 370 is coupled with the control box 90. However, a patient need not necessarily decouple the data logger 370 from the coil head 354 in order to couple the data logger 370 with the control box 90. Moreover, pressure measurements can be recorded in the memory 280 during the night in addition to or as an alternative to recording such measurements during the day, and pressure measurements can be recorded twenty-four hours a day. In that way, timing of pressure measurement taking and recordation need not be limited to the daytime only.

As described above, the data logger 370 can receive, store, and communicate data relating to pressure within the distension system. However, the data logger 370 can receive, store, and/or communicate a variety of other types of data. By way of non-limiting example, the data logger 370 can also receive, process, store, and/or communicate data relating to temperature, EKG measurements, eating frequency of the patient, the size of meals eaten by the patient, the amount of walking done by the patient, etc. It will therefore be appreciated by those skilled in the art that the data logger 370 can be configured to process received data to create additional data for communicating to the control box 90. For example, the data logger 370 can process pressure data obtained via the coil head 354 to create data indicative of the eating frequency of the patient. It will also be appreciated by those skilled in the art that the data logger 370 can include additional components to obtain non-pressure data. For example, the data logger 370 can include a pedometer or accelerometer (not shown) to obtain data relating to the amount of walking done by the patient. Data obtained by such additional components can be stored in the memory 280 and communicated to the control box 90 in a manner similar to pressure data. The data logger 370 can also include components for obtaining data to be factored in with internal pressure measurements to account for effects of various conditions on the pressure. For example, the data logger 370 can include a barometer for measuring atmospheric pressure. In some embodiments, the data logger 370 includes an inclinometer or similar device to determine the angle at which the patient is oriented (e.g., standing, lying down, etc.), which can be factored into pressure data to account for hydrostatic pressure effects caused by a patient's orientation. Alternatively, an inclinometer or other device for obtaining non-pressure data can be physically separate from the data logger 370 (e.g., implanted). Still other types of data, ways in which such data may be obtained, and ways in which such data may be used will be appreciated by those skilled in the art.

It will also be appreciated by those skilled in the art that one or more embodiments described herein can enable health care providers or others to use pressure data as a feedback mechanism to identify, train, and/or prescribe dietary advice to a patient. Such a feedback mechanism can provide data or otherwise be used in multiple ways. For example, pressure feedback can be obtained when a patient swallows a particular food portion, and based on such pressure feedback, the patient can be advised or taught to eat smaller portions, larger portions, or portions equal to the portion tested. Of course, a food portion so prescribed can be tested by evaluating pressure feedback obtained when the patient swallows the prescribed food portion, such that a food portion prescription may be refined through reiteration. As another example, a patient can test desired foods for appropriateness based on pressure feedback together with portion size and/or based on any other parameters. It will also be appreciated by those skilled in the art that continuous pressure data monitoring can be used locally and/or remotely to enable portion size monitoring, food consistency monitoring (e.g., liquids vs. solids), eating frequency, and/or other patient activities.

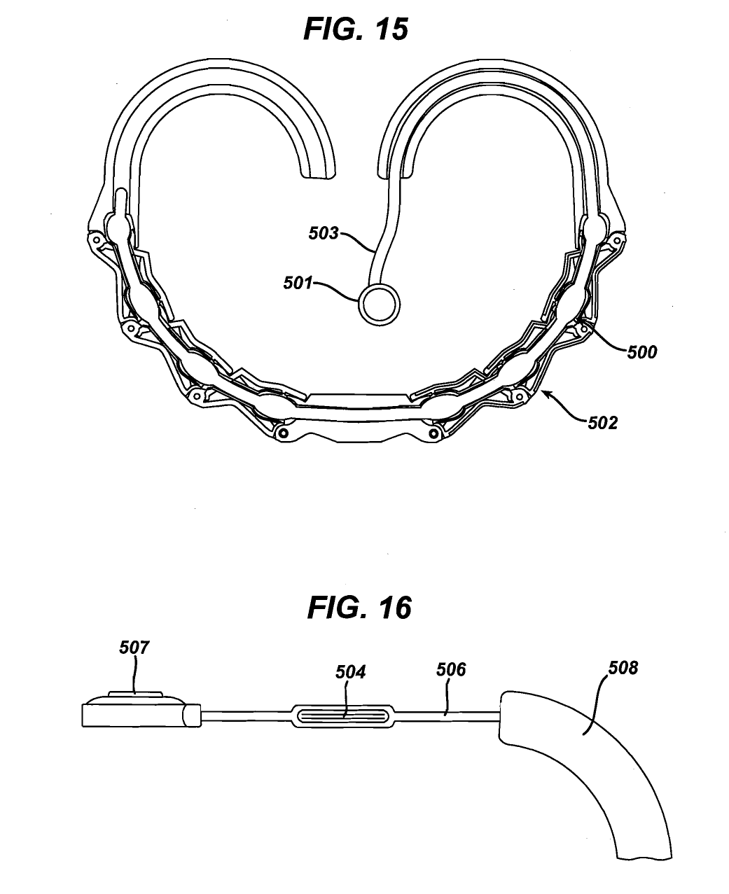

While embodiments described above include the use of the pressure sensor 62 within the sensor housing 60 removably joined to the catheter 50, a pressure sensor can be located elsewhere within a patient. For example, the pressure sensor 62 could be included in the port housing 30. In another embodiment, shown in FIG. 15, a pressure sensor 500 can be located within a gastric coil 502, such as in an inflatable portion of gastric coil 502. To the extent that the gastric coil 502 includes a resilient portion and a non-resilient portion, the pressure sensor 500 can be secured to either or neither of the resilient portion or non-resilient portion. In any case, the pressure sensor 500 can sense and communicate fluid pressure within the gastric coil 502 before, during, and after fluid is added to or withdrawn from gastric coil 502 via an injection port 501 and a catheter 503. The pressure sensor 500 can be used when a pump (not shown) or any other device is used to adjust pressure within the gastric coil 502.

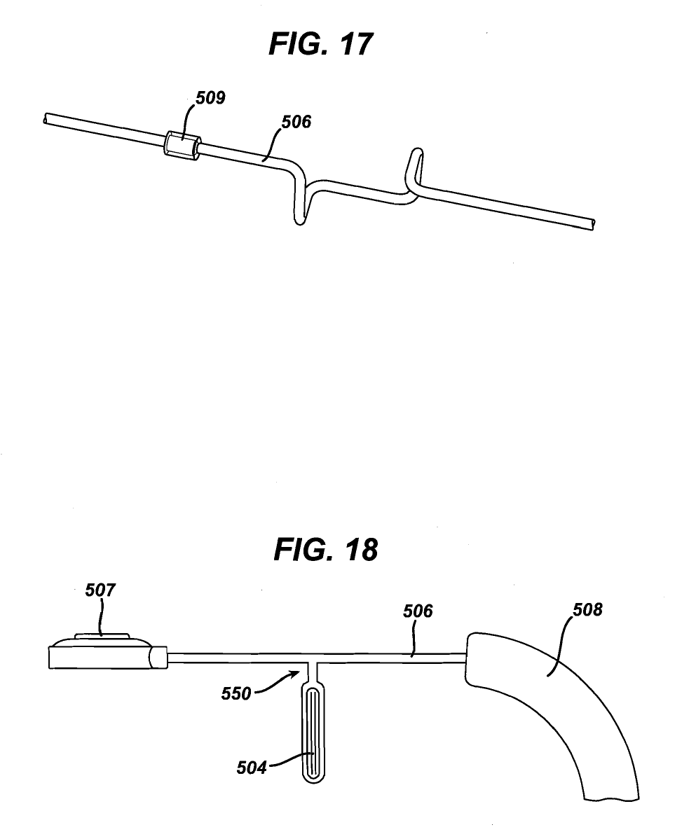

Alternatively, as shown in FIG. 16, a pressure sensor 504 can be located within a catheter 506 positioned between a gastric coil 508 and a port 507, pump, reservoir, or other device in fluid communication with the catheter 506. As another variation, an example of which is shown in FIG. 17, a pressure sensor 509 can be fixedly secured in-line with a catheter 506, while not residing within catheter 506.

Yet another variation is shown in FIG. 18, which illustrates a catheter 506 having a “T”-shaped intersection 550. A pressure sensor 504 is disposed in the arm of the “T”-shaped intersection 550 that is perpendicular to the catheter 506 and is in fluid communication with the catheter 506. In one embodiment, the “T”-shaped intersection 550 is integrally formed with the catheter 506 (as shown). In another embodiment, the “T”-shaped intersection 550 is a separate component joined to the catheter 506 (e.g., using barbed connectors, etc.). Other suitable ways in which the “T”-shaped intersection 550 can be provided will be appreciated by those skilled in the art. Similarly, other ways in which a pressure sensor 504 can be provided within, in-line with, or adjacent to the catheter 506 will be appreciated by those skilled in the art.

In yet another embodiment (not depicted), a pressure sensor can be located at the interface of an injection port and a catheter, and/or at the interface of a gastric coil and a catheter. Still other suitable locations for a pressure sensor will be appreciated by those skilled in the art, including but not limited to any location in or adjacent to the fluid path of a gastric coil system. In addition, a pressure sensor can be positioned within (e.g., against an inner wall of) a gastric coil, a catheter. Other suitable configurations for housing a pressure sensor within or adjacent to a coil, catheter, or buckle will be appreciated by those skilled in the art.

In another embodiment, a plurality of pressure sensors can be used. For example, a gastric coil system can include a pressure sensor within a gastric coil in addition to a pressure sensor within a catheter that is in fluid communication with the gastric coil. Such a plurality of pressure sensors can provide an indication of how well fluid pressure is distributed among components of a gastric coil system. Such a plurality of pressure sensors can also provide greater accuracy in pressure readings, reduce the likelihood of catheter obstruction (e.g., pinching) affecting pressure reading, reduce effects of hydrostatic pressure changes from patient movement, and/or provide one or more other results. Any system that includes a plurality of pressure sensors can include a pressure sensor in a port housing and/or a pressure sensor external to the patient (e.g., a pressure sensor in a syringe or in a pressure sensor portion coupled with a syringe), in addition to any of the implanted pressure sensors described above. Furthermore, a device such as an internal or external inclinometer (or a substitute therefore) may be used to determine the angle at which the patient and/or the internal portion is oriented (e.g., standing, lying down, etc.), which may be factored into pressure data sensed by one or more sensors to account for hydrostatic pressure effects caused by a patient's orientation. Such a factor (or any other factor) may be accounted for prior to or in conjunction with the rendering of a pressure reading.

A person skilled in the art will appreciate that the present invention has application in conventional endoscopic and open surgical instrumentation as well application in robotic-assisted surgery.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

Any patent, publication, application or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed:

1. An implant for placement within a hollow body organ, said implant comprising:
a. an implantable distension device having an undeployed shape for delivery within a hollow body and one or more deployed shapes for implantation therein;
b. said implantable distension device having sufficient rigidity in its deployed shape to exert an outward force against an interior of the hollow body so as to bring together two substantially opposing surfaces of said hollow body;
c. a powered means for changing the deployed shape of said implantable distension device while implanted within said hollow body; and
d. an implantable sensing device in communication with the implantable distension device and configured to sense a parameter related to the implantable distension device and to communicate said parameter to a filter, said filter transmits a selected portion of said parameter to a data storage device.

2. The implant of claim 1, wherein the parameter includes at least one of pressure, pulse count, pulse width, and amplitude ph, temperature, acceleration, composition, hormonal content.

3. The implant of claim 1, wherein the sensing device is also configured to communicate data to an external device when said external device telemetrically provides at least some power to the sensing device.

4. The implant of claim 1, wherein the sensing device is configured to discard data that substantially equals a nominal value.

5. The implant of claim 1, wherein the sensing device is configured to communicate a selected portion of data based on whether the data includes a value within a defined range of values.

6. The implant of claim 1, wherein the sensing device is configured to compare data with a nominal value.

7. The implant of claim 1, wherein the sensing device is configured to store the selected portion of data prior to communication of the selected portion of data to the external device.

8. The implant of claim 1, wherein the selected portion of data is compressed prior to communication of the selected portion of data to the external device.

9. An implant for placement within a hollow body organ, said implant comprising:
a. an implantable distension device having an undeployed shape for delivery within a hollow body and one or more deployed shapes for implantation therein;
b. said implantable distension device having sufficient rigidity in its deployed shape to exert an outward force against an interior of the hollow body so as to bring together two substantially opposing surfaces of said hollow body;
c. a powered means for changing the deployed shape of said implantable distension device while implanted within said hollow body;
d. an implantable sensing device in communication with the implantable distension device and configured to sense a parameter related to the implantable distension device and to communicate said parameter to a filter, said filter transmits a selected portion of said parameter to a data storage device; and
e. a processor configured to determine whether to store any of the sensed pressure data prior to communicating any of the sensed pressure data to an external reading device.

10. The implant of claim 9, further comprising an external storage mechanism that is configured to store sensed pressure data and to communicate stored pressure data to an external device.

11. The implant of claim 10, wherein the external storage mechanism is also configured to be removably attached to the patient.

12. The implant of claim 9, wherein the processor is configured to have a download of stored data to the external reading device triggered when the external reading device is moved in proximity of the implantable pressure sensing device.

13. The implant of claim 9, wherein the implantable pressure sensing device includes the processor.
